(19) United States
(12) Patent Application Publication
(10) Pub. No.: US 2020/0077974 A1
(43) Pub. Date: Mar. 12, 2020

(54) ULTRASOUND AND MULTISPECTRAL PHOTOACOUSTIC SYSTEMS AND METHODS FOR BRAIN AND SPINAL CORD IMAGING THROUGH ACOUSTIC WINDOWS

(71) Applicant: Wayne State University, Detroit, MI (US)

(72) Inventors: Kamran Avanaki, Detroit, MI (US); Juri G. Gelovani, Detroit, MI (US)

(73) Assignee: Wayne State University, Detroit, MI (US)

(21) Appl. No.: 16/566,212

(22) Filed: Sep. 10, 2019

Related U.S. Application Data
(60) Provisional application No. 62/728,962, filed on Sep. 10, 2018.

(51) Int. Cl. A61B 8/08; A61B 8/06; A61B 5/00

(52) U.S. Cl. CPC A61B 8/0866; A61B 8/085; A61B 8/0808; A61B 8/4483; A61B 5/0095; A61B 5/0035; A61B 8/5207; A61B 8/06

(57) ABSTRACT

Methods and systems are described for multispectral, non-invasive, and real-time assessment to diagnose hemorrhaging and/or hypoxia through PA imaging through an acoustic window defined in a body, such as through transfontanelle PA imaging of a neonatal infant brain. Such methods and systems include transmitting a plurality of ultrasound (US) waves and light between the probe device and the acoustic window, converting, via the probe device, a plurality of reflected US waves and generated PA waves into a plurality of US and PA signals, displaying in real-time on an US machine communicatively coupled to the probe device one or more images of the brain or spinal cord through the acoustic window, such as the neonatal infant brain through the fontanelle, and diagnosing at least one of one or more hemorrhages and hypoxia in the neonatal infant brain at least partially based on the one or more images.

### CROSS-REFERENCE TO RELATED APPLICATIONS

The present disclosure claims the benefit of U.S. Provisional Patent App. No. 62/728,962, filed Sep. 10, 2018, entitled “ULTRASOUND AND MULTISPECTRAL PHOTOACOUSTIC SYSTEMS AND METHODS FOR BRAIN AND SPINAL CORD IMAGING THROUGH ACOUSTIC WINDOWS,” the entirety of which is incorporated by reference herein.

### TECHNICAL FIELD

The present disclosure generally relates to ultrasound (US) and multispectral photoacoustic (PA) systems and methods for brain assessment through an acoustic window defined in a body of an individual and, more specifically, to US and PA systems and methods for multispectral, non-invasive, and real-time assessment of neonatal hemorrhage and/or hypoxia in a neonatal infant brain of a neonatal infant through use of transfontanelle imaging.

### BACKGROUND

Transfontanelle ultrasound (US) imaging is a diagnostic brain imaging method that may be used in premature infants or those that are younger than 6 months. The skull bones of such infants have not completely fused together and form openings between them termed fontanelles. Open fontanelles in such infants provide acoustic windows in the skull through which an US beam may pass. Transfontanelle US imaging may be used in diagnosis of neurological complications of premature birth, including: subarachnoid hemorrhage, intraventricular hemorrhage, subependimal hemorrhage, subdural hemorrhage, intracerebral hemorrhage, as well as hypoxic brain injuries resulting from a lack of oxygen to the neonatal infant brain. However, the accuracy of such transfontanelle US imaging may be limited, as sonography may underdiagnose at least low grade intraventricular and/or subarachnoid hemorrhages.

For example, transfontanelle US imaging has low sensitivity and low specificity for detection of hemorrhages that are less than 5 mm, diffuse subarachnoid hemorrhage in the neonatal infant brain, and small cerebral or extra-axial hemorrhages. Further, transfontanelle US imaging may be limited in detection of low concentration of blood in cerebrospinal fluid (“CSF”) such as less than 5% as well as detection of a vasogenic edema following a hemorrhage or ischemia/reperfusion injury. Further, transfontanelle US imaging may be limited by low sensitivity and low specificity for detection of non-cystic white matter lesions.

Accordingly, alternative systems and methods to provide accurate diagnoses of hemorrhage and/or hypoxia in a neonatal infant brain with increased sensitivity and specificity through non-invasive imaging are desired.

### BRIEF SUMMARY

According to the subject matter of the present disclosure, a system for a neonatal infant brain assessment of a neonatal infant through transfontanelle imaging may include one or more processors, one or more memory modules communicatively coupled to the one or more processors, an ultrasound machine comprising a display and communicatively coupled to the one or more memory modules, a probe device communicatively coupled to the ultrasound machine, and machine readable instructions stored in the one or more memory modules. The probe device may include a transducer, a soft tip configured to direct light through a diffusive material and to a fontanelle of the neonatal infant, and a fiber optic configuration. The machine readable instructions may cause the system to perform at least the following when executed by the one or more processors: transmit a plurality of ultrasound (US) waves and light from the probe device toward a neonatal infant brain upon placement adjacent a fontanelle of the neonatal infant, wherein the transmitted light comprises laser pulses configured to be tunable based on a change in wavelength; receive, into the probe device, a plurality of reflected US waves and generated PA waves; convert, via the probe device, the plurality of reflected US waves and generated PA waves into a plurality of US and PA signals; transmit, via the probe device, the plurality of US and PA signals to the ultrasound machine; generate one or more images of the neonatal infant brain at least partially based on the US and PA signals in real-time; and display the one or more images on the display of the ultrasound machine.

In accordance with one embodiment of the present disclosure, a method for multi-parametric, non-invasive, and real-time assessment of neonatal hemorrhage in a neonatal infant brain of a neonatal infant may include: positioning a probe device near a fontanelle of the neonatal infant, wherein the probe device is communicatively coupled to an ultrasound (US) machine and one or more processors and the probe device comprises a transducer, a soft tip configured to direct light through a diffusive material and into the fontanelle of the neonatal infant, and a fiber optic configuration of an optical fiber assembly, and transmitting a plurality of US and photoacoustic (PA) signals from the probe device toward the neonatal infant brain through the fontanelle. The method may further include receiving, into the transducer of the probe device, a plurality of reflected US waves and generated PA waves, converting, via the probe device, the plurality of reflected US waves and generated PA waves into a plurality of US and PA signals, and transmitting, via the probe device, the received plurality of US and PA signals to the US machine, generating one or more images of brain tissue and blood flow in the neonatal infant brain based on the US and PA signals, displaying in real-time the one or more images via the US machine, and diagnosing at least one of one or more hemorrhages and hypoxia at least partially based on the one or more images.

In accordance with one other embodiment of the present disclosure, an ultrasound (US) and multispectral photoacoustic (PA) probe device for transfontanelle imaging through a fontanelle of an infant brain may include: a US transducer comprising one of a linear array transducer and a curved array transducer, a soft tip comprising a diffusive material, an internal surface housing the diffusive material, a thin gold coating on the internal surface, the soft tip configured to direct light through the diffusive material to the fontanelle, and an inside shell and an outside shell defining a shell space therebetween, wherein the inside shell is configured to be positioned exterior to the transducer. The probe device may further include a fiber optic configuration comprising a plurality of fiber optic cables configured in one of a square configuration and a honeycomb configuration to transmit light as a series of laser pulses from the laser transmitting light in a wavelength range of from about 532 nm to about 1064 nm to a penetration depth of up to about 10 cm and direct light at a bending angle directed toward the fontanelle. At least a portion of the fiber optic configuration may be disposed in the shell space.

These and additional features provided by the embodiments described herein will be more fully understood in view of the following detailed description, in conjunction with the drawings.

### BRIEF DESCRIPTION OF THE SEVERAL VIEWS OF THE DRAWINGS

The embodiments set forth in the drawings are illustrative and exemplary in nature and are not intended to limit the subject matter defined by the claims. The following detailed description of specific embodiments of the present disclosure can be best understood when read in conjunction with the following drawings, where like structure is indicated with like reference numerals and in which:

FIG. 1 illustrates an isometric view of a ultrasound (US) and multispectral photoacoustic (PA) transfontanelle imaging probe for imaging of a neonatal infant brain, incorporating aspects of the present disclosure;

FIG. 2 is an exploded view of the example probe of FIG. 1, according to aspects of the present disclosure;

FIG. 3 is a partial cross-sectional, isometric view of the probe of FIG. 1 illustrating a pair of fiber optic assemblies housed within the probe, according to aspects of the present disclosure;

FIG. 4 is a partial cross-sectional, isometric view of the probe of FIG. 1 illustrating a single of fiber optic assembly housed within the probe and disposed around a curved array transducer, according to aspects of the present disclosure;

FIG. 5 is a partial cross-sectional, isometric view of the probe of FIG. 1 illustrating the curved array transducer housed in a shell of the probe, according to aspects of the present disclosure;

FIG. 6 is a partial cross-sectional, isometric view of the probe of FIG. 1 illustrating the shell of the probe configured to house the curved array transducer, according to aspects of the present disclosure;

FIG. 7 illustrates an isometric side view of another ultrasound (US) and multispectral photoacoustic (PA) transfontanelle imaging probe for imaging of a neonatal infant brain, incorporating aspects of the present disclosure; and

FIG. 8 is an exploded view of the probe of FIG. 7 including a linear array transducer and illustrating a pair of fiber optic assemblies housed within the probe, according to aspects of the present disclosure;

FIG. 9 is a cross-sectional, isometric rear view of the probe of FIG. 7, according to aspects of the present disclosure;

FIG. 10 is a cross-sectional, isometric side view of the probe of FIG. 7 illustrating a fiber optic assembly housed within the probe adjacent to the linear array transducer, according to aspects of the present disclosure;

FIG. 11 is a cross-sectional, isometric bottom view of the probe of FIG. 7, according to aspects of the present disclosure;

FIG. 12 is a schematic top plan view of the probe of either FIG. 1 or FIG. 7 placed at the center of a fontanelle providing an acoustic window to a neonatal infant brain of a neonatal infant, according to aspects of the present disclosure;

FIG. 13 is a schematic side plan view of the probe of either FIG. 1 or FIG. 7 disposed above tissue defining the center of a fontanelle of a neonatal infant, according to aspects of the present disclosure;

FIG. 14A is a schematic view of a square configuration of a fiber optic assembly of the probe of either FIG. 1 or FIG. 7, according to aspects of the present disclosure;

FIG. 14B is a schematic view of a honeycomb configuration of a fiber optic assembly of the probe of either FIG. 1 or FIG. 7, according to aspects of the present disclosure;

FIG. 15A is a fiber optic configuration including one row at one side of a transducer of the probe of either FIG. 1 or FIG. 7, according to aspects of the present disclosure;

FIG. 15B is another fiber optic configuration including one row on each side of a transducer of the probe of either FIG. 1 or FIG. 7, according to aspects of the present disclosure;

FIG. 15C is another fiber optic configuration including two rows on each side of a transducer in a square configuration of a fiber optic assembly of the probe of either FIG. 1 or FIG. 7, according to aspects of the present disclosure;

FIG. 15D is another fiber optic configuration including three rows on each side of a transducer in a honeycomb configuration of a fiber optic assembly of the probe of either FIG. 1 or FIG. 7, according to aspects of the present disclosure;

FIG. 16 schematically illustrates an example imaging system for implementing computer and software based methods to utilize the probe of either FIG. 1 or FIG. 7, according to one or more embodiments shown and described herein;

FIG. 17 illustrates a schematic view of an optical assembly of a laser system and a laser light coupling including a fiber optic bundle, according to one or more embodiments shown and described herein;

FIG. 18 illustrates a schematic isometric and partially exposed view of the laser light coupling of FIG. 17, according to one or more embodiments shown and described herein;

FIG. 19 illustrates example US and PA imaging of cerebrospinal fluid (“CSF”) within a neonatal infant brain to diagnose subarachnoid, subependymal, intraventricular, and intracerebral hemorrhages, according to one or more embodiments shown and described herein;

and

FIG. 20 illustrates an example calibration curve representative of a light fluence profile with respect to depth, according to one or more embodiments shown and described herein.

### DETAILED DESCRIPTION

The present disclosure relates to systems and methods to improve clinical care of neonatal infants by using brain and/or spinal cord PA imaging to provide direct information regarding oxygen saturation in the brain and/or spinal cord of an individual through an acoustic window opening in the overlaying bones. As a non-limiting example, the present disclosure relates to systems and methods to improve clinical care of a neonatal infant by using PA imaging through the fontanelle (transfontanelle) to provide direct information regarding oxygen saturation of the brain of the infant. Also, transfontanelle PA imaging allows for detection of intracerebral, intraventricular, subdural, and epidural hemorrhages, as well as hypoxic brain injuries resulting from a lack of oxygen to the infant brain. The systems and methods described herein further permit visualization of infant brain tissue and brain vasculature to estimate oxygen saturation and diagnose hemorrhages in the visualized brain imaging.

Premature and/or underweight neonatal infants may suffer from intraventricular hemorrhage that leads to excessive bleeding into the ventricles inside the infant brain. Using a rapid, portable, and minimally or non-invasive diagnostic method to diagnose such hemorrhaging assists to improve a future quality of life for such infants. A transfontanelle multispectral PA and US imaging probe as described herein allows for such non-invasive functional imaging of the infant brain to assist with such diagnoses. The probe may be used for bedside monitoring to detect hemorrhages in neonatal infants that may be suffering from complications due to premature birth, for example. Such a probe may be used to deliver a thermally-optimal laser energy through the fontanelle of the infant that accounts for differences in laser fluence. Light fluence may be approximately 2.4 times greater inside the infant brain through transfontanelle illumination, as described herein in an embodiment at a wavelength of the laser pulse of 570 nm, as compared to skull illumination at the same depth. Such increased light fluence through the fontanelle (transfontanelle illumination) may be due to a lower attenuation coefficient through the fontanelle resulting in increased penetration as compared to skull illumination.

In a non-limiting example, the probe uses an illumination scheme in combination with tissue attenuation to create a sophisticated light fluence profile with respect to depth. Light fluence is indicative of scalar irradiance incidence on a sphere of a unit cross-section per unit surface area of the sphere and per unit time. The scalar irradiance as light fluence is a time integrated quantity that is a rate quantity corresponding to exposure to light. Normalized PA signals at each depth for the probe are approximately proportional to a fluence at each depth. An inverse of a fluence function of the probe may be used for fluence decay compensation at a specified depth. By way of example, and not as a limitation, strong attenuation of a laser may be observed in brain tissue due to scattering and absorption of light within tissue layers that may lead to inhomogeneous optical distribution. As a result, generated PA signals may not be accurate and may need to be compensated. Therefore, a fluence decay compensation approach may be utilized for such a correction and calibration of the generated PA signals. Light decay in an MRI-based reference model of the brain of the infant may be performed using Monte Carlo simulations to obtain a simulated profile. A PA signal profile is then divided by the simulated profile. A resultant signal, and corresponding resultant image, may then be fluence compensated. For such fluence compensation, initially, a simulation-based 3D fluence distribution map is generated based on an optical fiber configuration and optical absorption of the brain tissue. Next, structural information based on a brain tissue model may be determined and the attenuation coefficient due to scattering phenomenon may be extracted. A combination of the scattering coefficient model and the simulated fluence map may then be applied to compensate the fluence decay in an original three-dimensional PA map of the actual imaging study of the brain of the infant.

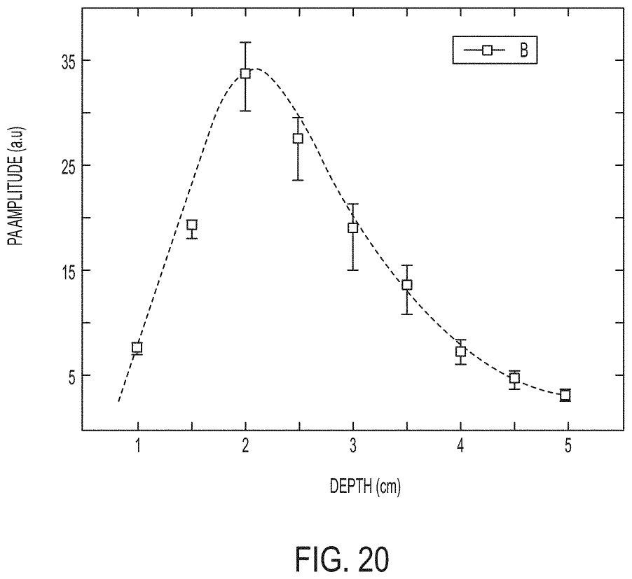

In a fluence compensation experiment, a calibration curve as shown in FIG. 20 was generated through imaging through a probe as described herein and a system 300 of FIG. 16 as described in greater detail below of a heparinized sheep blood. By way of an example and not a limitation, the heparinized sheep blood was placed in a thin capillary suspended in a solution in a plastic container utilized as an experimental acoustic window 30 (FIG. 16) to mimic absorption and scattering coefficients of brain tissue. For example, the thin capillary included a 3/32″ inner diameter and a 1/32″ wall thickness, and the solution was an INTRALIPID-ink solution as available through SIGMA ALDRICH, USA with 97%/0.01% concentrations. Blood was supplied by a 150 mL syringe and moved through use of an automatic syringe pump at a fixed flow speed of 100 mL/hour. Constant flow allowed the blood and saline to maintain homogeneity. Cubic spline interpolations were applied to the data to smooth results and reduce error. A generated calibration curve as shown in FIG. 20 plots the results of imaging of the blood B with respect to PA amplitude and depth. For example, PA amplitude is plotted on the y-axis as representative of fluence in a range of about 0 to 40 a.u., and depth is plotted on the x-axis in a range of from about 0 cm to 5 cm. The calibration curve for the experimental results shows a peak light fluence of approximately 35 a.u. at a depth of approximately 2.1 cm.

Such a probe further determines blood vessel oxygen saturation levels using a dual-wavelength model, and hemorrhage size, location, and/or age may be detected using the probe. As a non-limiting example, an age of a hemorrhage may be determined based on determined quantitative concentrations of blood decay products such as bilirubin and biliverdin. In an embodiment, to determine an age of a hemorrhage, the PA imaging probe may be utilized to determine a concentration of different blood decaying components. Imaging may be performed at different wavelengths that correspond to high absorption coefficients specified for each of the components. The products and associated imaging wavelengths may be oxy-hemoglobin (HbO) at 578 nm, total hemoglobin (HbT) at 570 nm, bilirubin at 680 nm, and biliverdin at 440 nm. Imaging may be performed every ten minutes for about 2 hours. Decay in each imaged component may represent a decrement in the PA signal intensity at the specified wavelength. Based on acquired data from the imaging, the age of the hemorrhage may then be determined. Blood decay products in CSF may be measured through spectrometry such as through absorption of light of a certain wavelength by different products of hemoglobin decay such as bilirubin, biliverdin, and bilirubin oxidation products. Further, the probe may be used to detect a vessel rupture location using an exogenous contrast agent.

A high sensitivity of transfontanelle PA imaging in comparison to transfontanelle US imaging with respect to blood provides a manner to accurately diagnose hemorrhage and/or hypoxia in a neonatal infant brain with increased sensitivity and specificity through non-invasive imaging in a non-traumatic manner to the neonatal infant brain. Through the use of multiple wavelengths with such PA spectroscopy, tissue oxygen saturation (SO₂) may be measured and used to diagnose tissue hypoxia and provide evidence of oxygen availability in a circulatory system of the neonatal infant without time delay as the oxygen saturation may be quantified based on hemoglobin as an endogenous contrast agent. By contrast, certain other hypoxia-related imaging modalities such as positron emission tomography (PET) involve an injection of contrast, leading to a time delay of image acquisition and SO₂ maps of hypoxic regions. PET further potentially exposes a neonatal infant brain of a neonatal infant to prohibitively high levels of ionizing radiation. The methods and systems described herein are directed to a non-ionizing approach that is safe and effective for the neonatal infant.

A transfontanelle multispectral PA and US imaging probe as described herein for transfontanelle PA and US imaging combines and contrasts optical imaging and a spatial resolution of US imaging. Bodily substances have a specific optical absorption coefficient that is unique to endogenous chromospheres of cells or tissue. Substances that are to be imaged may be illuminated by a nanosecond pulsed laser of a specific wavelength at which the absorption coefficient is the highest. Photon absorption by absorbing substances cause a transient temperature change leading to a thermal expansion and a localized pressure change and acoustic waves that are detected by an ultrasonic transducer. PA imaging of the infant brain is based on an acoustic detection of optical absorption from tissue chromophores, such as oxy-hemoglobin (HbO) and deoxy-hemoglobin (HbR), which have differing absorption spectra for optical imaging. Such PA imaging is able to simultaneously provide high-resolution imaging of brain vasculature and hemodynamics. US imaging can image a blood flow rate in functionally active regions of the brain, while PA imaging may differentiate between oxygenated or deoxygenated blood and determinate a regional brain oxygen extraction fraction. The probe devices 100, 200 described herein may determine a measurement of oxygen saturation based on a PA signal difference between oxy-hemoglobin and deoxy-hemoglobin values of brain tissue illuminated through the fontanelle 10. Tissue oxygen saturation may be measured based on data from the one or more images and use of hemoglobin as an endogenous contrast agent, and an oxygen consumption in the neonatal infant brain may be estimated to diagnose tissue hypoxia based on the measured oxygen saturation.

By way of example and not as a limitation, US transducers coupled to a US machine may emit pulses of a frequency in a range of about 5 MHz to 10 MHz from a 128 element array in a non-ionizing manner toward a bodily substance and collect sound waves that bounce back to create and display images on the US machine. The US transducer may record changes in a pitch and direction of the bounced back acoustic waves to measure and display these waves as a real-time image. In PA imaging, non-ionizing laser pulses and/or radio frequency (RF) pulses may be used for thermos-acoustic imaging and delivered to the body substance such as biological tissue. A portion of the delivered energy is absorbed into the tissue and converted to heat that leads to a transient thermoelastic expansion and wideband ultrasonic emission, and the US transducer detects the generated ultrasonic waves as generated PA waves to create and generate images on the US machine. Optical absorption through the PA imaging is associated with physiological properties such as tissue oxygen saturation and hemoglobin concentration and may display physiological specific optical absorption contrast to form such images. Blood typically has a higher optical absorption than surrounding tissue and thus may act as an endogenous contrast for such PA imaging.

Thus, in combination, US imaging and PA imaging may generate highly accurate quantitative parametric images of blood flow/oxygen extraction as regional brain oxygen consumption. For example, as described above, an application of PA imaging for a neonatal infant assists with determining a tissue oxygen saturation (SO₂) measurement that may be used to diagnose hypoxia, for example, as well as for therapy planning and treatment monitoring.

A two-wavelength based oxygen saturation measurement set forth as Equation 1 below may be utilized to determine the SO₂ measurement.

$${SO}_{2} = - \frac{{ɛ}_{Hb}^{W 2}}{Δ {ɛ}_{Hb}^{W 2}} + \frac{{ɛ}_{Hb}^{W 1}}{Δ {ɛ}_{Hb}^{W 2}} \frac{{μ}_{a}^{W 2}}{{μ}_{a }^{W 1}} \qquad ( Equation 1 )$$

In Equation 1 above, εₓ is a molar extinction coefficient and μₐ is an absorption coefficient at a certain wavelength, which in the example above includes a first wavelength W₁ and a second wavelength W₂. The first wavelength W₁ may be at 570 nm, and the second wavelength W₂ may be at 578 nm. A ratio of absorption coefficient may be substituted with a ratio of a PA signal intensity.

Use of the probe devices described herein configured to provide transfontanelle US and multispectral PA imaging for detection of hemorrhages such as small intraventricular hemorrhages of less than 5 mm, intraparenchymal hemorrhages, and diffuse subarachnoid hemorrhages while further being able to detect a low concentration of blood in the CSF of less than about 5% while being able to measure the age, size, and/or location of the hemorrhage. As a non-limiting example, the probe device as described herein may be used to at least detect blood concentrations as low as 1.0% in the CSF, detect intraparenchymal hemorrhages of at least 1 mm in diameter and their age, and measure tissue oxygen saturation of up to a distance of about 6.0 cm, including up to a distance of about 4.5 cm, from a soft tip 107, 207 of the probe 100, 200 as described in greater detail below. In an embodiment, blood concentrations of less than 5% may be detected in the CSF of a subarachnoid of the neonatal infant. Blood in the CSF may be assessed at concentrations of as low as 1% of ventricular volume and concentrations of blood decay products may be assessed in a manner as described above with respect to determination of an age of a hemorrhage to estimate an age of an intraventricular bleed. Small intraparenchymal bleeds of about 1 mm may be accurately located and a determination of when the bleed occurred may be determined from the data acquired from the probe 100, 200.

Further, quantitative imaging of brain tissue oxygenation may be provided while being able to detect through such imaging leaky capillaries in white and grey matter lesions of the brain that disrupt a brain blood barrier to cause vasogenic edema. As a non-limiting example, the probe device as described herein may be used to at least measure tissue oxygen saturation up to a depth of a range between 2.0 cm to about 10.0 cm, such as 4.5 cm or 6.0 cm, as a non-limiting example from the scalp and detect leaky capillaries in white and grey matter lesions that result from hemorrhage or hypoxia/ischemia/reperfusion injury using a contrast agent. The contrast agent may be a methylene blue dye or Evans blue dye or other like contrast agent, which may be used with a wavelength of 665 nm or 605 nm, respectively, for example. In an embodiment, locations of the vasogenic edema caused by the disruption to the blood-brain barrier may be accurately mapped based on the retrieved data from the probe 100, 200.

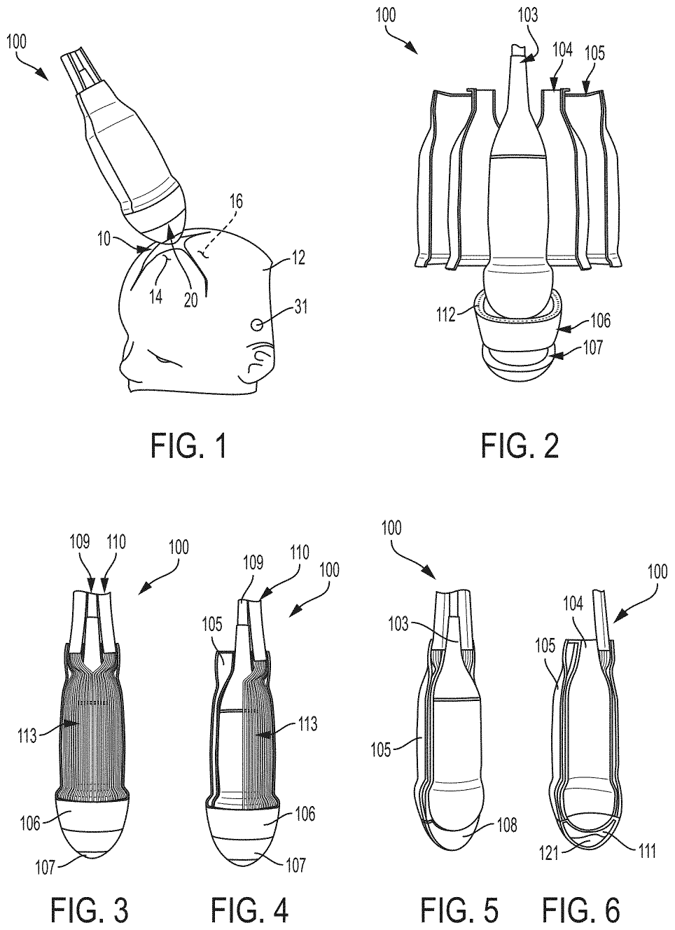

Referring initially to FIGS. 1-6, a probe device 100 is illustrated to provide laser light 20 for transfontanelle PA imaging with respect to a fontanelle 10 adjacent tissue 14 of an infant 12 to provide PA imaging of an infant brain 16. An example of such imaging of the infant brain 16 is shown in FIG. 19. In particular, FIG. 19 illustrates example US and PA imaging through an image 500 of cerebrospinal fluid (“CSF”) 502 within a neonatal infant brain to diagnose subarachnoid, subependymal, intraventricular, and intracerebral hemorrhages. The image 500 will be able to depict the CSF 502 as filled with blood, for example, as blood typically has a higher optical absorption than surrounding tissue. Imaging of the CSF 502 filled with blood through such an optical contrast image may be indicative of such a hemorrhage.

Referring to FIG. 2, an exploded view of the probe device 100 is illustrated including a curved array transducer 103, an inside shell 104, and outside shell 105, a base ring 106 including a plurality of fiber optic channels 112, and a soft tip 107. FIG. 3 illustrates an ultrasound data cable 109 and a pair of fiber optic cable bundles 110, each housing a plurality of fiber optic cables 113 respectively configured for receipt in the plurality of fiber optic channels 112 of the base ring 106. FIG. 4 illustrates a single fiber optic cable bundle 110 housed between the outside shell 105 and the inside shell 104 and disposed about the curved array transducer 103 that is housed by the inside shell 104. FIG. 5 illustrates the pair of fiber optic cable bundles 110 including the plurality of fiber optic cables 113 housed between the outside shell 105 and the inside shell 104 that houses the curved array transducer 103. FIG. 6 illustrates a single fiber optic cable bundle 110 including the plurality of fiber optic cables 113 housed between the outside shell 105 and the inside shell 104 without showing the curved array transducer 103 for illustrative purposes. In an embodiment, the soft tip 107 may include an internal surface 121 housing a diffusive material that is a light-diffusive material such as a diffusive US gel 108, the internal surface 121 coated with a thin gold coating 111, and be configured to project the laser light 20 from the plurality of fiber optic cables 113 of the probe device 100. Plurality of fiber optic channels 112 of the base ring 106 are configured to direct light from the housed plurality of fiber optic cables 113 through the diffusive material such as the diffusive US gel 108 and through the soft tip 107 to the acoustic window 30 such as the fontanelle 10. The soft tip 107 in combination with the diffusive material is thus configured to act as a diffuser to disperse and scatter the light from the plurality of fiber optic cables 113 to generate a uniform distribution of light for exposure to and absorption by the neonatal infant brain 12 over a larger area than a concentrated beam of light without such diffusion. Through such a uniform distribution of light, the neonatal infant brain 12 receives similar light intensity at different portions across the larger area exposed to the light and a more uniformly illuminated and homogenized light fluence profile for imaging of the neonatal infant brain 12.

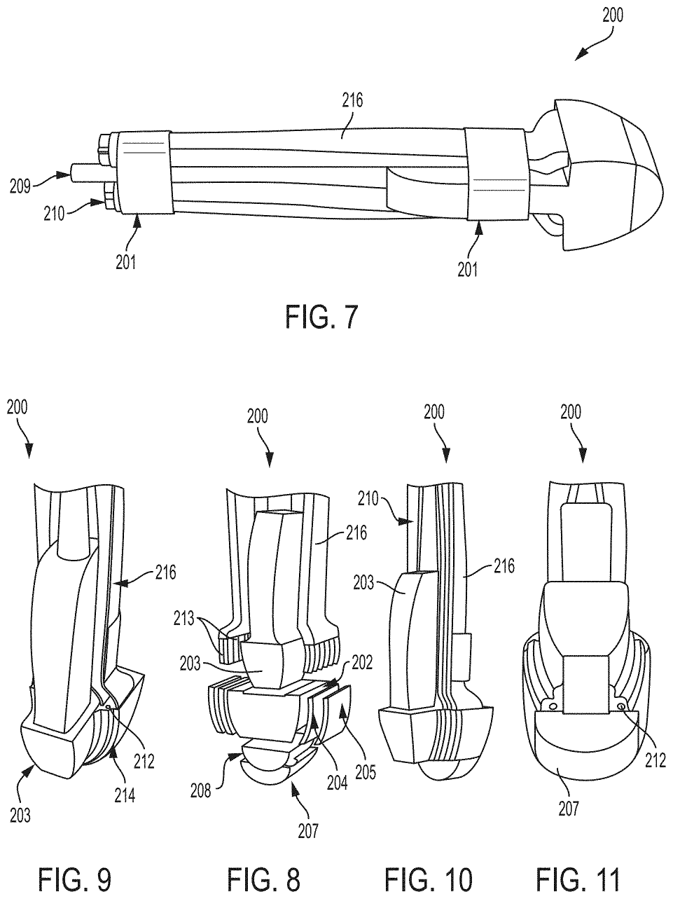

Referring to FIGS. 7-11, another example probe device 200 is illustrated to provide laser light 20 for transfontanelle PA imaging of the infant brain 16 through the fontanelle 10 of the infant 12. FIG. 7 illustrates an isometric side view of the probe device 200, which includes one or more flexible straps 201, a US data cable 209, a pair of fiber optic cable bundles 210, and a pair of fiber optic bundle housings 216 configured to house the pair of fiber optic cable bundles 210. The one or more flexible straps 201 are configured to be disposed about the pair of fiber optic bundle housings 216 and other components of the probe device 200 to hold the components together and may be made of a medical grade material.

FIG. 8 illustrates an exploded view of the probe device 200. A main body of the probe device may be a 3D printed polylactic acid (PLA). The probe device 200 illustrates a linear array transducer 203 housed between the pair of fiber optic bundle housings 216 and configured for receipt in a transducer housing 202. In an embodiment, the linear array transducer 203 may include 128 elements. The linear array transducer 203 may have a center frequency in a range of from about 5 MHz to 10 MHz, an element size including a height in a range of from about 5 mm to 8 mm and a width in a range of about 0.2 mm to about 0.3 mm, and give a penetration depth in a range of from about 2 cm to 6.5 cm and/or an axial resolution in a range of from about 350 μm to about 450 μm. In an embodiment, the probe device 100, 200 may give an axial resolution of 416 μm and a lateral resolution of 1.72 mm. The probe device 100, 200 may target depths in a range of from about 0.5 cm to about 5 cm, such as depths of 0.5 cm, 1.0 cm, 1.5 cm, 2 cm, 2.5 cm, 3 cm, 3.5 cm, 4 cm, 4.5 cm and 5 cm. As a non-limiting example, the linear array transducer 203 may have a center frequency of 5 MHz, have 128 elements with an element size including of a height of 7 mm and a width of 0.25 mm, and give a penetration depth of up to 10 cm, or 6 cm, or 4.5 cm and an axial resolution of approximately 400 μm.

A frequency of the probe device 100, 200 may be configured to allow imaging of the infant brain 16 down to a skull base with an axial resolution of less than about 500 μm at a penetration depth of approximately 5 cm or of approximately 4.5 cm. Applied wavelength may be in a range of from about 532 nm to about 1064 nm based on a penetration depth and optical energy requirement. A selected wavelength may keep the optical energy below an ANSI limit of 1064 nm to prevent burning to the infant brain or skull, for example. In an embodiment, a wavelength to measure concentrations of oxy-hemoglobin (HbO), deoxy-hemoglobin (HbR), and total hemoglobin (HbT) in deep brain structures of depths to around 4 cm to 5 cm may be 578 nm for HbO, 560 nm for HbR, and 570 nm for HbT. It is contemplated and within the scope of this disclosure that the metrics and/or materials described herein may be applied to either of the probe device 100 or the probe device 200.

An inside shell 204 is configured to be disposed adjacent each side wall of the transducer housing 202, and an outside shell 205 is configured to be disposed adjacent and outer to the inside shell 204. A diffusive material such as a diffusive US gel 208 is configured to be disposed beneath a bottom portion of the transducer housing 202, which bottom portion is disposed between and below the side walls of the transducer housing 202. A soft tip 207 is configured to be disposed below the diffusive gel 208, which is disposed between the soft tip 207 and the transducer housing 202. The soft tip 207 may be made of an Aqualene material, which has acoustic properties similar to the fontanelle 10 and to water to reduce acoustic impedance mismatch and keep attenuation minimal to negligible. As a non-limiting example, water may have an impedance of about 1.48 MRayl and an attenuation of about 0.054 dB/cm @ 5 MHz while the Aqualene material has an impedance of about 1.46 MRayl and an attenuation of about 2.8 dB/cm @ 5 MHz. The Aqualene material may further have material properties such as a density of about 0.92 g/cm³, a coefficient of thermal expansion of about 3×10⁻³ c⁻¹, a reference temperature of about 22° C., and a Poisson ratio of about 0.34. The Aqualene material may be translucent and not significantly impact light scattering and absorption. As a non-limiting example, the Aqualene material may be a translucent material with minimal to no light scattering, with only 0.2% light intensity reduction at a wavelength of 532 nm for a layer having a 2 mm thickness. In an embodiment, a thickness of the soft tip layer made of the Aqualene material may be 0.5 mm.

Each fiber optic bundle housing 216 configured to house fiber optic cable bundles 210 terminates in a plurality of fiber optic cables 213 forming the fiber optic cable bundles 210 that project from ends of the fiber optic bundle housing 216. The fiber optic cables 213 may be made of borosilicate glass. The fiber optic cables 213 for a pair of projecting rows disposed adjacent to each side wall of the transducer housing 202. Each projecting row projects from ends of the fiber optic bundle housing 216 such that each projecting fiber optic cable 213 is configured for receipt in a fiber optical cable channel 212 of a fiber optics projection housing 214. A first set of fiber optic cable channels 212 is formed between the inside shell 204 and each side wall of the transducer housing 202 and is configured for receipt of a first projecting row of fiber optic cables 213. A second set of fiber optic cable channels 212 is formed between the inside shell 204 and the outside shell 205 and is configured for receipt of a second projecting row of the fiber optic cables 213. The projecting rows of the fiber optic cables 213 project laser light through bottom openings of the fiber optic cable channels 212 through the diffusive material such as the diffusive US gel 208 to project light through the soft tip 207 toward an acoustic window 30 such as the fontanelle 10 of the infant 12. The soft tip 207 in combination with the diffusive material is thus configured to act as a diffuser to disperse and scatter the light from the plurality of fiber optic cables 213 to generate a uniform distribution of light for exposure to and absorption by the neonatal infant brain 12 over a larger area than a concentrated beam of light without such diffusion.

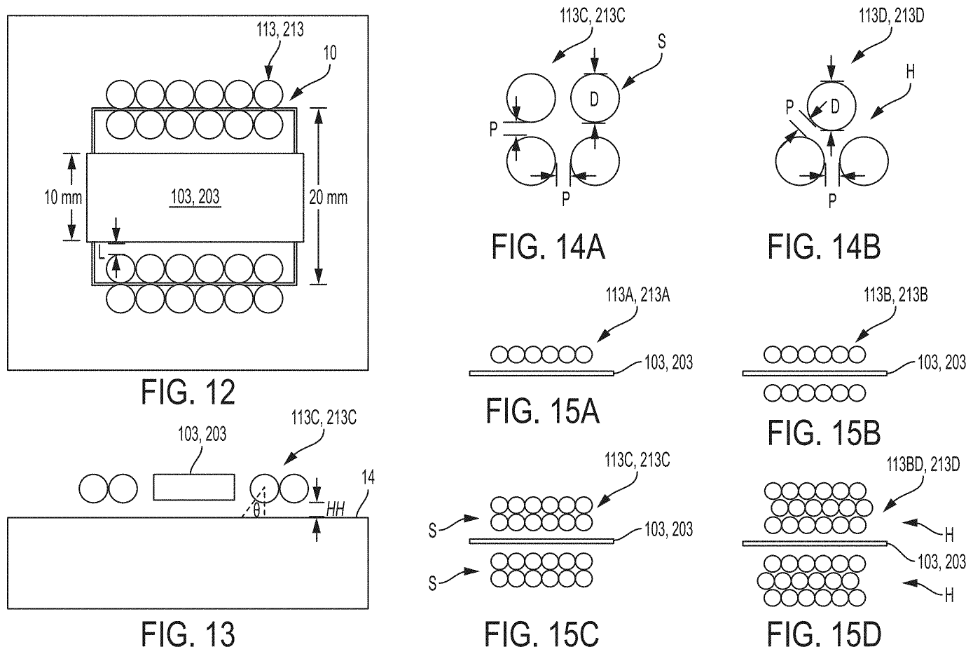

Referring to FIGS. 12-15D, the fiber optic cables 113, 213 may include fibers in one of a row configuration, square configuration S, or honeycomb configuration H. As a non-limiting example, the fibers may include 36 fibers. In an embodiment, the fibers are housed in a proximal end and reflect light through the soft tip 107, 207 as described herein and may be borosilicate glass optic fibers of a wide-bandwidth in a range of from about 500 nm to about 1300 nm that are each 2 m long. At an opposite distal end of the probe 100, 200, the fibers may be bundled in a holder. An additional optical fiber may be placed in front of an energy meter such as available from GENTEC INTEGRA, USA to monitor pulse-to-pulse fluctuations to assist with optical energy normalization.

In an embodiment, each fiber 113, 213 may have a fiber diameter D of about 2 mm, a numerical aperture of 0.2, a bending angle θ of about 30 degrees, a vertical distance HH to a tissue 14 of 6 mm, and distance L between a first row of fiber optic cables 113, 213 and the US transducer 103, 203 of 8 mm. In an embodiment, a plurality of US waves and light are transmitted from the probe device 100, 200 toward the fontanelle 16 of the neonatal infant 12 when the probe device 100, 200 is positioned within a distance range from the neonatal infant brain, which distance range is the vertical distance HH. The distance range may be from about 5 mm to about 10 mm from the fontanelle 10.

The fontanelle 10 may be a 2 cm by 2 cm (i.e., 20 mm by 20 mm) range, and the transducer 103, 203 may have a width of about 1 cm (i.e., 10 mm). The average size of anterior fontanelle may be 2 cm×2 cm. The detection part of the probe device 100, 200 may be 2 cm×1 cm. While the optical fiber configuration may be slightly outside of the fontanelle window, the bending angle θ directed toward the fontanelle window allows for illumination of the 2 cm×2 cm fontanelle window, for example.

The tip 107, 207 of the probe device 100, 200 may be made of the Aqualene material, which as described above has acoustic properties similar to the fontanelle 10 and water to reduce acoustic impedance mismatch and keep attenuation minimal to negligible. PA imaging through the fontanelle 10 makes detection of US waves possible without high acoustic scattering otherwise caused by the skull. In an embodiment, the light source illuminating the area of the fontanelle 10 should have an intensity distribution close to a uniform distribution such that an optimal amount of light energy may be delivered to the fontanelle 10 without the intensity exceeding an allowable threshold. Thermal parameters for brain, skull, and scalp for the infant 12 may respectively be the following: densities of 1.081, 1.807, and 1.084 gr/cm³; specific heat capacities of 3.68, 1.3, and 3.39 J/Kg; thermal conductivities of 0.53, 0.5, and 0.3 W/mK, and volumetric blood perfusions of 0.009, 0.0025, and 0.0035 s⁻¹.

In an embodiment, a width of the probe device 100, 200 may be less than or equal to 4 cm. Referring to FIG. 12, a top view of a US transducer 103, 203 placed at a center of the fontanelle 10 is illustrated with optical fibers 113, 213 located at sides of the US transducer 103, 203 to act as light sources. A distance L between a first row of fiber optic cables 113, 213 and the US transducer 103, 203 may be a range of from about 5 mm to 10 mm to accommodate in a range of transducer widths in a range of from about 10 mm to 20 mm. In an embodiment, the distance L between the first row of fiber optic cables 113, 213 and the US transducer 103, 203 may be 5 mm, 6 mm, 7 mm, 8 mm, 9 mm, or 10 mm.

FIG. 13 illustrates a side view of a distance HH between the fiber optic cables 113, 213 and the tissue 14 along with the bending angle θ for each fiber of the fiber optical cables 113, 213. FIG. 14A illustrates the fiber diameter D along with a fiber positioning P between a square configuration S of fiber optic cables 113C, 213C, which fiber positioning P between adjacent fibers may be about 0.5 mm. FIG. 14B illustrates the fiber diameter D along with a fiber positioning P between a honeycomb configuration H of fiber optic cables 113D, 213D, which fiber positioning P between adjacent fibers may be about 0.5 mm. In embodiments, the fiber diameter D may be 0.5 mm, 1.0 mm, 1.5 mm, 2.0 mm, or 2.5 mm, and the numerical aperture may be 0.1, 0.2, 0.3, 0.4, or 0.5 for a wide range of both glass and/or plastic optical fibers. For example, a graded index plastic optical fiber made of Poly(methyl methacrylate) (PMMA) may be used to deliver light. The bending angle θ may be 0, 10, 20, 30, 40, 50, or 60 degrees as an angle of an optical fiber axis with respect to normal to a surface of the tissue 14. The distance HH may be 5, 6, 7, 8, 9, or 10 mm. In an embodiment, for D of 0.5, 1.0, 1.5, 2.0, and 2.5 mm respectively, a maximum number of rows for fiber optic cables 113, 213 may be 9, 6, 5, 4, and 3 and/or the number of fibers in a row may be 19, 13, 10, 8, and 7.

FIG. 15A illustrates a single row configuration for fiber optic cables 113A, 213A showing one row at one side of the transducer 103, 203. FIG. 15B illustrates a dual single row configuration for fiber optic cables 113B, 213B showing one row at each side of the transducer 103, 203.

FIG. 15C illustrates a square configuration S for fiber optic cables 113C, 213C showing two rows at each side of the transducer 103, 203. As a non-limiting example, the fiber optic configuration forms the square configuration S such that at least two rows of fiber optic cables 113, 213 form a square lattice pattern of aligned rows for the square configuration S and are disposed on each side of the transducer 103, 203.

FIG. 15D illustrates a honeycomb configuration H for fiber optic cables 113D, 213D showing three rows at each side of the transducer 103, 203. As a non-limiting example, the fiber optic configuration forms the honeycomb configuration H such that at least three rows of fiber optic cables 113, 213 forma hexagonal lattice pattern of alternating rows for the honeycomb configuration H and are disposed on each side of the transducer 103, 203. In embodiments, a honeycomb configuration H may provide more uniform optical intensity distribution as compared to a square configuration S with otherwise similar parameters.

The different configurations of FIGS. 15A-15D involve different light intensity profiles that are able to be determined to assess configuration performance. For example, an array M representative of a homogeneity and performance of a fiber optic configuration is defined in Equation 2 below.

$$M = \frac{{E}_{fontanelle}}{{E}_{0}} \times \frac{{E}_{mean}}{{E}_{ma x}} \qquad ( Equation 2 )$$

In Equation 2 above, E_{fontanelle} is representative of a total energy deposited inside an area of the fontanelle 10, E₀ is representative of a total output light energy from the probe device 100, 200 in a single pulse, Eₘₑₐₙ is representative of a mean energy in the area of the fontanelle 10, and Eₘₐₓ is representative of a maximum value of deposited energy. A higher M value is representative of a more uniform intensity profile. A value Nₚᵢₓₑₗ may be representative of a number of pixels inside the area of the fontanelle 10 when using the probe device 100, 200. In an embodiment, Nₚᵢₓₑₗ may equal 201×201=40401 pixels. A deposited energy E may be determined from I×da, where I is representative of intensity and da is representative of an area of each pixel. In an embodiment in which all light is incident on a single pixel, a used term may be

$$\frac{{E}_{fontanelle}}{{N}_{pixel }} .$$

An illuminated surface may be divided into pixels with the area da=0.1 mm×0.1 mm to create a grid, and a number of photons hitting the surface may be integrated over each pixel to generate a deposited energy E and an intensity I, where I=E/da.

Through a simulation study, a value of M=0.7696 was found for a fiber optic configuration including four rows of a honeycomb configuration H at each side of a transducer 103, 2013 having a width of 10 mm, where the optical fibers had a diameter D of 2 mm, a numerical aperture of 0.2, a bending angle θ of 30 degrees, an HH of 8 mm, and an L of 5 mm. Further, a value of M=0.6543 was found for a fiber optic configuration including five rows of a honeycomb configuration H at each side of a transducer 103, 2013 having a width of 10 mm, where the optical fibers had a diameter D of 1.5 mm, a numerical aperture of 0.5, a bending angle θ of 40 degrees, an HH of 5 mm, and an L of 5 mm.

In an embodiment, and referring to FIG. 16, a system 300 may be an imaging system. The system 300 may include a communication path 310, one or more processors 304, a memory component 306, a probe device 100, 200 including a transducer, a storage or database 308, an imaging module 302, a server 320, network interface hardware 322, a network 324, a laser system 312, and an optical parametric oscillator (OPO) 314. Laser light is sent from the laser system 312 through the OPO 314 and through the fiber optic cable bundles 110, 210 for receipt and projection through respective probe devices 100, 200. The laser system 312 may include an OPOTEK PHOCUS MOBILE laser head. The leaser head may be a dimension of 109.2 cm in length, 48.3 cm in width, and 76.2 cm in depth and be a class 4 laser system. The OPO 314 range may be from about 690 nm to about 950 nm with a pulse length in a range of from about 4 ns to about 6 ns with a repetition rate of 10 Hz or 20 Hz and a maximum output of 130 mJ/pulse. The laser system 312 may further include a power supply and chiller.

Additionally, the system 300 includes an acquisition system 318 including the imaging module 302 communicatively coupled to the transducer probe device 100, 200 through components such as network interface hardware 322 and wires of the communication path 310. The acquisition system 318 may be communicatively coupled to the imaging module 302 through either a wired or wireless connection. The acquisition system 318 may be a US real-time data acquisition system including 128 channels that may submit US waves and receive reflected US signals back through transducers 103, 203 of the probe devices 100, 200 with respect to the fontanelle 10 of the infant 12. The acquisition system 318 may be an acquisition system as commercially available through VANTAGE SYSTEMS or VERASONICS, USA. The acquisition system 318 may be triggered directly from the power supply of the laser system 312.

Further, the one or more processors 304 of the system 300 includes a FPGA based control unit communicatively coupled to a laser of the laser system 312 and the acquisition system 318. The FPGA may be high speed at about 100 MHz or faster and may be a center timing unit in the system 300. The laser system 312 is communicatively coupled to the OPO 314 that converts an input laser wave with a frequency into two output waves of lower frequency. The laser system 312 and the OPO 314 may operate at 30 Hz and utilize real-time pulse energy monitoring. The transducer probe device 100, 200 may send waves and/or light such as tunable laser pulses to and receive signals back from an acoustic window 30 schematically disposed below the transducer probe device 100, 200. The acoustic window 30 may be, for example, a fontanelle 10 of an infant brain 16 of an infant 12, a cranial opening made after a craniotomy that creates a surgical opening into a skull of a person, or a spinal opening formed by a laminectomy.

By way of example, and not as a limitation, the transducer probe device 100, 200 may be used for intraoperative and postoperative imaging of a brain through a surgically created hole such as a burr hole 31 (FIG. 1) and/or through other surgically-created cranial openings including, but not limited to, surgically-induced cranial bone openings and surgically-induced vertebral laminar openings. In such scenarios, one or more surgically-created openings may serve as one or more acoustic windows 30 that respectively provide an opening for light penetration generated by the probe device 100, 200 and penetration of ultrasound (acoustic) waves generated and/or detected by the probe device 100, 200, similar to how a fontanelle 10 present in the skull of a neonatal infant 12 as described herein provides an acoustic window opening for acoustic and light penetration to and from the infant brain 16. The probe device 100, 200 may thus assist a surgeon such as a neurosurgeon in defining an extent of surgical intervention that may be required for, as a non-limiting example, removal of intracranial hemorrhage and/or definition of tumor margins for resection. Further, the probe device 100, 200 may be used with an imaging system such as the system 300 for post-operative monitoring of a status of a surgical cavity post evaluation of a hemorrhage, to monitor for re-bleeding within the surgical cavity, to assess the completeness of tumor resection, and/or to monitor tumor recurrence. In an embodiment, the probe device 100, 200 may be placed in contact with skin over a resected bone defect in the skull, such as a burr hole 31 (FIG. 1) serving as an acoustic window 30 (FIG. 16), and may be used with the system 300 to image the brain of an individual whether an infant or older such as an adult. In another embodiment, the probe device 100, 200 may be placed in contact with skin over the resected laminectomy defect, serving as another type of an acoustic window 30, and may be used with the system 300 to image the spinal cord. The probe device 100, 200 may be used with ultrasound (US) and near-infrared (NIR) light conducting gel and the system 300 for such imaging. A photoacoustic (PA) image based on PA signals received through use of the probe device 100, 200 may be overlaid onto a received US image based on reflected US signals received through use of the probe device 100, 200 to generate one or more images for such imaging.

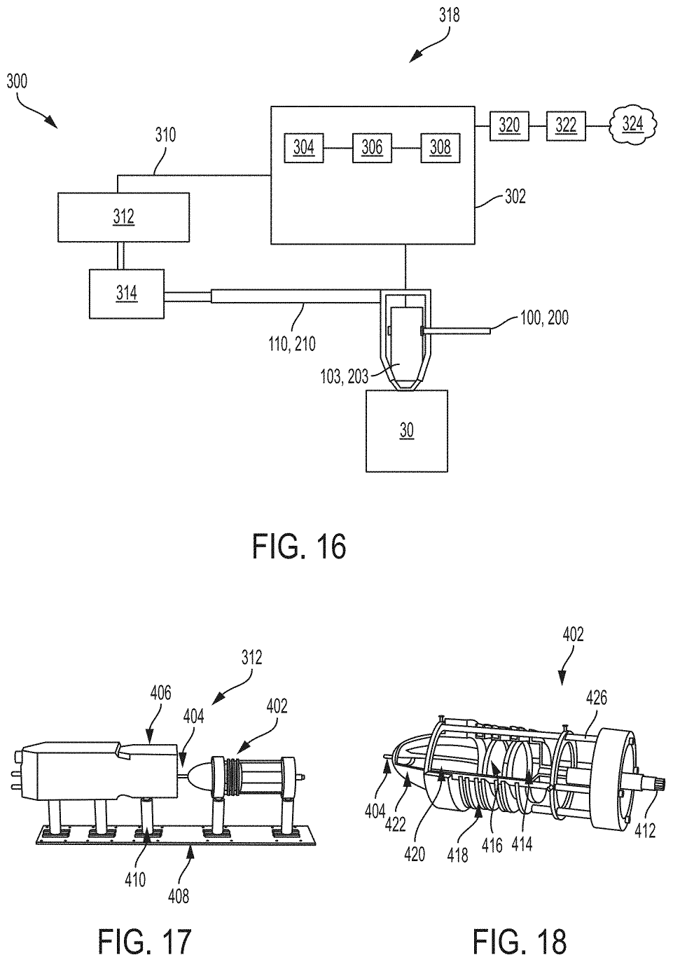

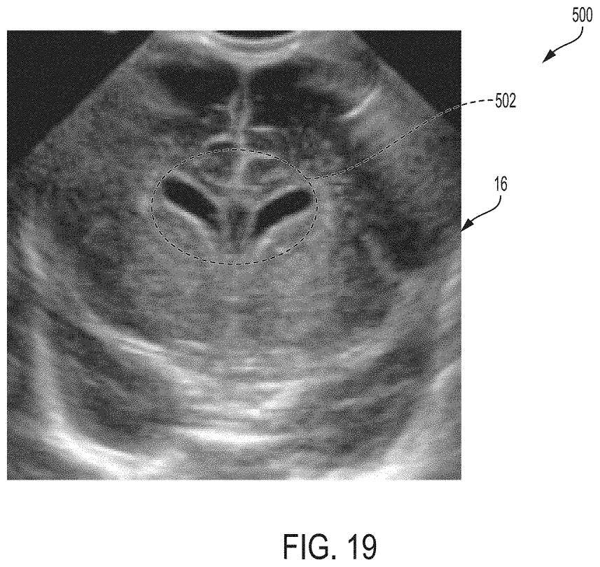

FIG. 17 depicts an embodiments of the laser system 312 including laser light coupling optics 402, a laser light 404, a laser system head 406, an optical breadboard 408, and a plurality of optical posts 410 to connect and hold the laser light coupling optics 402 and the laser system head 406 to the optical breadboard 408. FIG. 18 illustrates a partially exposed view of the laser light coupling 402. The laser light coupling 402 includes a fiber optic cable bundle 412 configured to communicate with the fiber optic cable bundles 110, 210 of the probe devices 100, 200. The fiber optic cable bundles 412 may be comprises of borosilicate glass. The fiber optic cable bundles 412 may include 36 fibers to 40 fibers, have a length of 2 m, and may accommodate a wide-bandwidth of from about 500 nm to about 1300 nm. The laser light coupling 402 further includes a convex lens 414, a diffuser 416, a plurality of spacers 418 to separate internal components such as the convex lens 414 and the diffuser 416 together configured for homogenization and focusing of light at a distal end of an optical fiber bundle. The convex lens 414 may be an achromatic double lens AC254-040-B as commercially available from THORLABS, USA. The convex lens 414 may have a diameter of 1 in and a focal depth of 40 mm. The laser light coupling 402 further includes a parabolic reflector 420 and a gold coating 422 at an end through which the laser light 404 is received. The parabolic reflector 420 may be a 3D printed PLA including the thin gold coating 422. The cage housing defining the laser light coupling optics 402 and housing the convex lens 414 and the diffuser 416 may also be made from a 3D printed PLA. The laser system 312 may include a tunable laser such as a QUANTA-RAY PRO ND:YAG laser using a repetition rate of 30 Hz and a pulse width of 7 ns, for example. The OPO 314 may be a GWU VERSA SCAN used to tune the wavelength, and the tunable laser may have a range of from about 450 nm to about 1100 nm with a maximum energy of 100 mJ. In an embodiment, laser energy may be measured using an energy meter such as one commercially available through GENTEC INTEGRA, USA that may be connected to a laptop to acquire and store an energy log for further normalization. Software used for reading the energy data may be PC GENTEC-EO software.

In another embodiment, a probe device as described herein may include an illumination configuration that allows a high energy deposition to the brain tissue of the infant 12 through consideration of a larger illumination area than the 2 cm×2 cm fontanelle area described herein even though part of the larger illumination area would be on the infant skull. As the skull of a neonatal infant is thin at about 0.8 mm to 2 mm and is composed of thin plates of compact and not spongy bone, a high optical energy deposition efficiency may be utilized with such an illumination configuration. The probe device with the described illumination configuration may include a parabolic, mirror-like, 3D printed optical fiber holder as, for example, a dome, that is configured to hold approximately 70 borosilicate glass optical fibers placed equidistantly and having a diameter of about 3 mm. In an embodiment, the dome is a 3D printed, parabolic-mirror-like that includes a silvered, inner reflective surface, and light exiting the laser of the laser system 312 passed through the dome for light delivery. A utilized pump laser output may be 500 mJ/cm² at 1064 nm. The system may include 50% fiber delivery efficiency, and a diameter adaption between the laser and a distal end of the fiber bundle of about 7 cm². Further, 100 mJ/cm² may be yielded on the scalp at below the ANSI limit at 1064 nm. A numerical aperture of 0.48 may be used, and a fiber configuration may produce a homogenized optical field on the surface of the scalp of the infant 12 when passed through a cushion-like water balloon with a very thin membrane layer. In embodiments, illumination of the fontanelle 10 may occur at an average of 15 mJ/cm² at 570 nm.

In operation, and in an embodiment, an optical fiber assembly comprised of the plurality of fiber optic cables 113, 213 of the probe device 100, 200 is communicatively coupled to a laser of the laser system 312, a plurality of US waves are transmitted from the transducer 103, 203 of the probe device 100, 200 as a series of sound wave signals, and light is transmitted from the optical fiber assembly as a series of laser pulses from the laser. Reflected US waves and generated PA waves (i.e., US waves generated from the absorption of light in the brain and resultant thermal expansion to generate US waves) are received by the probe 100, 200. The transducer 103, 203 of the probe 100, 200 converts the received reflected US waves and generated PA waves into respective US and PA signals. One or more images are generated of brain tissue and blood flow in the neonatal infant brain 16 based on the US and PA signals, and one of a hemorrhage and hypoxia in the neonatal infant brain 16 is diagnosed at least partially based on the one or more images.

As a non-limiting example, a system 300 for a neonatal infant brain assessment of a neonatal infant through transfontanelle imaging may include one or more processors 304, one or more memory modules such as a memory component 306 communicatively coupled to the one or more processors 304, an ultrasound machine comprising a display and communicatively coupled to the one or more memory modules, a probe device 100, 200 communicatively coupled to the ultrasound machine, and machine readable instructions stored in the one or more memory modules. The probe device 100, 200 may include a transducer 103, 203, a soft tip 107, 207 configured to direct light through a diffusive material such as diffusive US gel 108, 208 and to a fontanelle 10 of the neonatal infant 12, and a fiber optic configuration made of an arrangement of optical fibers 113, 213. The machine readable instructions may cause the system 300 to perform at least the following when executed by the one or more processors 304: transmit a plurality of ultrasound (US) waves and light from the probe device 100, 200 toward a neonatal infant brain 16 upon placement adjacent a fontanelle 10 of the neonatal infant 12, wherein the transmitted light are laser pulses configured to be tunable based on a change in wavelength. The instructions may further cause the system 300 to receive, into the probe device 100, 200, a plurality of reflected US waves and generated PA waves; convert, via the probe device 100, 200, the plurality of reflected US waves and generated PA waves into a plurality of US and PA signals; transmit, via the probe device 100, 200, the plurality of US and PA signals to the ultrasound machine; generate one or more images of the neonatal infant brain at least partially based on the US and PA signals in real-time; and display the one or more images on the display of the ultrasound machine.

The probe devices using multispectral PA and US imaging systems as described herein may provide improved, clinically relevant, diagnostic information over neonatal US imaging systems, improve point-of-care diagnosis at an infant beside, lead to an early postnatal intervention and improved management of infant hemorrhages, and be a cost-effective and portable diagnostic system and imaging method for neonatal infants with a potential for hemorrhaging. Further, the PA and US probe devices described herein use only light and sound, which are both non-invasive and non-ionizing, to continuously monitor a neonatal infant brain in a safe and effective manner.

A signal may be “generated” by direct or indirect calculation or measurement, with or without the aid of a sensor.

For the purposes of describing and defining the present invention, it is noted that reference herein to a variable being a “function” of (or “based on”) a parameter or another variable is not intended to denote that the variable is exclusively a function of or based on the listed parameter or variable. Rather, reference herein to a variable that is a “function” of or “based on” a listed parameter is intended to be open ended such that the variable may be a function of a single parameter or a plurality of parameters.

It is also noted that recitations herein of “at least one” component, element, etc., should not be used to create an inference that the alternative use of the articles “a” or “an” should be limited to a single component, element, etc.

It is noted that recitations herein of a component of the present disclosure being “configured” or “programmed” in a particular way, to embody a particular property, or to function in a particular manner, are structural recitations, as opposed to recitations of intended use. More specifically, the references herein to the manner in which a component is “configured” or “programmed” denotes an existing physical condition of the component and, as such, is to be taken as a definite recitation of the structural characteristics of the component.

It is noted that terms like “preferably,” “commonly,” and “typically,” when utilized herein, are not utilized to limit the scope of the claimed invention or to imply that certain features are critical, essential, or even important to the structure or function of the claimed invention. Rather, these terms are merely intended to identify particular aspects of an embodiment of the present disclosure or to emphasize alternative or additional features that may or may not be utilized in a particular embodiment of the present disclosure.

For the purposes of describing and defining the present invention it is noted that the terms “substantially” and “approximately” are utilized herein to represent the inherent degree of uncertainty that may be attributed to any quantitative comparison, value, measurement, or other representation. The terms “substantially” and “approximately” are also utilized herein to represent the degree by which a quantitative representation may vary from a stated reference without resulting in a change in the basic function of the subject matter at issue.

Having described the subject matter of the present disclosure in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Further, it will be apparent that modifications and variations are possible without departing from the scope of the present disclosure, including, but not limited to, embodiments defined in the appended claims. More specifically, although some aspects of the present disclosure are identified herein as preferred or particularly advantageous, it is contemplated that the present disclosure is not necessarily limited to these aspects.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

What is claimed is:

1. A system for a brain or spinal cord assessment of an individual through acoustic window imaging comprising:
one or more processors;
one or more memory modules communicatively coupled to the one or more processors;
an ultrasound machine comprising a display and communicatively coupled to the one or more memory modules;
a probe device communicatively coupled to the ultrasound machine, the probe device comprising a transducer, a soft tip configured to direct light through a diffusive material and to a fontanelle of the neonatal infant, and a fiber optic configuration; and
machine readable instructions stored in the one or more memory modules that cause the system to perform at least the following when executed by the one or more processors:
transmit a plurality of ultrasound (US) waves and light from the probe device toward a brain or spinal cord through an acoustic window defined as an opening to the brain or spinal cord of the individual, wherein the transmitted light comprises laser pulses configured to be tunable based on a change in wavelength;
receive, into the transducer of the probe device, a plurality of reflected US waves and generated PA waves;
convert, via the probe device, the plurality of reflected US waves and generated PA waves into a plurality of US and PA signals;
transmit, via the probe device, the plurality of US and PA signals to the ultrasound machine;
generate one or more images of the neonatal infant brain at least partially based on the US and PA signals in real-time; and
display the one or more images on the display of the ultrasound machine.

2. The system of claim 1, wherein the soft tip is made of a translucent material that has acoustic properties similar to water to reduce acoustic impedance mismatch and has an impedance of about 1.46 MRayl and an attenuation of about 2.8 dB/cm @ 5 MHz, and an internal surface of the soft tip comprises a thin gold coating.

3. The system of claim 1, wherein the instructions to transmit a plurality of US waves and light from the probe device comprise instructions to transmit the plurality of US waves and light form the probe device toward a neonatal infant brain of a neonatal infant upon placement adjacent a fontanelle of the neonatal infant for transfontanelle imaging of the neonatal infant brain.

4. The system of claim 3, further comprising machine readable instructions to:
determine a measurement of oxygen saturation based on a PA signal difference between oxy-hemoglobin and deoxy-hemoglobin values of brain tissue illuminated through the fontanelle.

5. The system of claim 3, wherein the plurality of US waves and light are transmitted from the probe device toward the fontanelle of the neonatal infant when the probe device is positioned within a distance range from the neonatal infant brain.

6. The system of claim 5, wherein the distance range is from about 5 mm to about 10 mm from the fontanelle.

7. The system of claim 1, wherein the fiber optic configuration comprises a square configuration such that at least two rows of fiber optic cables forming a square lattice pattern of aligned rows for the square configuration are disposed on each side of the transducer.

8. The system of claim 1, wherein the fiber optic configuration comprises a honeycomb configuration such that at least three rows of fiber optic cables forming a hexagonal lattice pattern of alternating rows for the honeycomb configuration are disposed on each side of the transducer.

9. The system of claim 1, wherein:
the fiber optic configuration comprises an optical fiber assembly communicatively coupled to a laser, and the optical fiber assembly comprises 36 fibers.

10. The system of claim 9, wherein:
the plurality of US waves are transmitted from the probe device as a series of sound waves.

11. The system of claim 9, wherein:
the light transmitted from the optical fiber assembly as a series of laser pulses signals from the laser.

12. The system of claim 1, wherein the transducer is one of a linear array transducer or a curved array transducer, each comprising an inside shell and an outside shell defining a shell space therebetween configured to house at least a portion of the fiber optic configuration.

13. A method for multispectral, non-invasive, and real-time assessment of neonatal hemorrhage in a neonatal infant brain of a neonatal infant, the method comprising:
positioning a probe device near a fontanelle of the neonatal infant, wherein the probe device is communicatively coupled to an ultrasound (US) machine and one or more processors, wherein the probe device comprises a transducer, a soft tip configured to direct light through a diffusive material and into the fontanelle of the neonatal infant, and a fiber optic configuration of an optical fiber assembly;
transmitting a plurality of US waves and light from the probe device toward the neonatal infant brain through the fontanelle;
receiving, into the transducer of the probe device, a plurality of reflected US waves and generated PA waves;
converting, via the probe device, the plurality of reflected US waves and generated PA waves into a plurality of US and PA signals;
transmitting, via the probe device, the plurality of US and PA signals to the US machine;
generating one or more images of brain tissue and blood flow in the neonatal infant brain based on the reflected US and PA signals;
displaying in real-time the one or more images via the US machine; and
diagnosing at least one of one or more hemorrhages and hypoxia at least partially based on the one or more images.

14. The method of claim 13, further comprising:
measuring tissue oxygen saturation based on data from the one or more images and use of hemoglobin as an endogenous contrast agent; and
estimating an oxygen consumption in the neonatal infant brain adjacent the fontanelle to diagnose tissue hypoxia based on the measured oxygen saturation.

15. The method of claim 14, wherein measuring tissue oxygen saturation based on data from the one or more images comprises:
determining a measurement of oxygen saturation from the one or more images based on a PA signal difference between oxy-hemoglobin and deoxy-hemoglobin values of brain tissue illuminated through the fontanelle.

16. The method of claim 13, wherein diagnosing one of a hemorrhage and hypoxia at least partially based on the one or more images comprises detecting blood concentrations of less than 5% in CSF of a subarachnoid of the neonatal infant.

17. The method of claim 13, wherein diagnosing one of a hemorrhage and hypoxia at least partially based on the one or more images comprises diagnosing hemorrhages comprising at least one of small intraventricular hemorrhages of less than 5 mm, intraparenchymal hemorrhages, and diffuse subarachnoid hemorrhages.

18. The method of claim 13, further comprising:
measuring through the probe device tissue oxygen saturation up to a depth of 4.5 cm; and
detecting at least partially based on the one or more images leaky capillaries in white and grey matter lesions of the neonatal infant brain that result from at least one of hemorrhage and hypoxia disrupt a brain blood barrier to cause vasogenic edema.

19. The method of claim 13, wherein:
the optical fiber assembly is communicatively coupled to a laser;
the plurality of US waves are transmitted from the transducer of the probe device as a series of sound waves; and
the light is transmitted from the optical fiber assembly as a series of laser pulses from the laser.

20. An ultrasound (US) and multispectral photoacoustic (PA) probe device for transfontanelle imaging through a fontanelle of an infant brain, wherein the probe device comprises:
a US transducer comprising one of a linear array transducer and a curved array transducer;
a soft tip comprising a diffusive material, an internal surface housing the diffusive material, a thin gold coating on the internal surface, the soft tip configured to direct light through the diffusive material to the fontanelle;
an inside shell and an outside shell defining a shell space therebetween, wherein the inside shell is configured to be positioned exterior to the US transducer; and
a fiber optic configuration comprising a plurality of fiber optic cables configured in one of a square configuration and a honeycomb configuration to transmit light as a series of laser pulses from a laser transmitting light in a wavelength range of from about 532 nm to about 1064 nm to a penetration depth of up to about 10 cm and direct light at a bending angle directed toward the fontanelle, wherein at least a portion of the fiber optic configuration is disposed in the shell space.
